(19) United States
(12) Patent Application Publication
(10) Pub. No.: US 2009/0299177 A1
(43) Pub. Date: Dec. 3, 2009

(54) Risk Stratification of Patients with Chronic Myocardial Infarction

(75) Inventors: Raghoottama Pandurangi, St. Charles, MO (US); Raghavan Rajagopalan, St. Peters, MO (US); Mary Dyszlewski, Creve Coeur, MO (US); Robert Carretta, Des Peres, MO (US); Joseph Pierro, Chesterfield, MO (US)

Correspondence Address: Mallinckrodt Inc., 675 McDonnell Boulevard, HAZELWOOD, MO, 63042, US

(73) Assignee: Mallinckrodt Inc., Hazelwood, MO (US)

(21) Appl. No.: 12/476,769

(22) Filed: Jun. 2, 2009

Related U.S. Application Data
(60) Provisional application No. 61/058,291, filed on Jun. 3, 2008.

(51) Int. Cl. A61B 6/00; C07F 13/00

(52) U.S. Cl. 600/431; 534/14

(57) ABSTRACT

The present invention is directed to novel radiopharmaceuticals, which can be used to image myocardium. In addition, the present invention is directed to methods for risk stratification of patients who have suffered at least one myocardial infarction.

### CROSS REFERENCE TO RELATED APPLICATIONS

This application claims the benefit of U.S. Provisional Application Ser. No. 61/058,291 filed Jun. 3, 2008.

### BACKGROUND

1. Field of the Invention

The present invention generally relates to novel radiopharmaceutical compounds and methods for risk stratification of patients who have suffered a myocardial infarction.

2. Background

Heart failure is a major health problem in the U.S. where nearly 400,000 new cases appear annually. Although an estimated 5 million patients suffering from acute chest pain are admitted to emergency departments each year, a high proportion of these patients are later found not to have required hospitalization. Yet there are also a significant number of heart attack patients who are mistakenly discharged from the emergency department. Such diagnostic errors are a major cause of emergency department malpractice claims. Predicting the onset of subsequent heart failure and establishing a suitable treatment protocol is important in the management of myocardial infarction (MI) patients.

Chronic heart failure appears most commonly in patients with previous myocardial infarction (MI) who develop ischemic cardiomyopathy (ICM), the leading cause of death in the U.S. The pathophysiology of cardiac failure in post-MI patients has been attributed to the slow, but chronic formation of fibrous tissue in unwanted areas of dysfunctional myocardium, which eventually impairs contractile function of the entire myocardium. The histopathology of ICM in explanted failing human hearts in post-MI patients reveals the presence of several abnormalities such as perivascular/interstitial fibrosis remote to a primary MI site, scarred segment of infarcted myocardium, and microscopic scars. This remote fibrotic tissue plays a major role in the adverse cardiac remodeling of post-MI patients.

Tissue repair is a fundamental property of vascular tissues. Following an injury to an organ, macrophages and fibroblasts are recruited to the site of injury where repair process begins by phenotypical transformation of fibroblasts to myofibroblasts (myoFbs) that play a central role in fibrogenesis at site of repair. Fibrous tissue formation is an integral part of the tissue repair and leads to stable scar formation that preserves the structural integrity of the organ. MyoFbs have been proposed to play a retractile role in wound contraction. They replace the lost cells through the deposition of extra cellular matrix (ECM), and impart contractility to scar tissue tone through the formation of fibrous tissue consisting of collagens I and III.

Angiotensin II (AngII, A-II) is the active biological octapeptide of the systemic renin angiotensin system, which is a circulating humoral system responsible for blood pressure regulation and salt-water homeostasis. AngII exerts its effects through binding to specific receptors. There are two major subtypes of AngII receptors: type 1 (AT1) and type 2 (AT2). Most of the physiological actions of AngII are regulated through AT1. Recently, in addition to the classical role, AngII has attracted attention for its novel role as a growth factor. Growth-modulating effects of these receptors were reported in cardiac vascular endothelial cells, smooth muscle cells, and fibroblasts, where AT1 caused cell proliferation and synthesis of extracellular matrix proteins, whereas AT2 acted in an anti-proliferative manner (see, e.g., Fujiyama et al., Circ Res. 2001; 88:22-29).

The up-regulation of angiotensin II (A-II) receptor that occurs after MI incident is an important event in the establishment of stable scar formation. Once the stable scar is established, myofibroblasts undergo apoptosis; the A-II expression is decreased to the background level, constituting successful tissue repair. On the other hand, when the tissue repair fails, the proliferation of myofibroblasts remote to and at the site of MI leads to unbridled tissue repair resulting in unabated organ fibrosis (known as adverse remodeling). Continued formation of fibrous tissue, i.e. excessive scarring, is the major cause of organ failure.

The myocardium of post MI patients typically exhibit ‘low blood flow’ zone and ‘no blood flow zone.’ The cells in the former region are still viable and can be resurrected with proper intervention, whereas in the latter, the damage is permanent and no revival is possible. The perfusion-based radiopharmaceuticals currently used in imaging post MI patients cannot differentiate between these two zones, in that both regions appear as “cold spots” in radioscintigraphic scans. Therefore, these agents tend to overestimate myocardial viability, and many patients fall mistakenly into a “low viability” category. As a result, patients who are in real need of invasive treatment procedures such as revascularization, or implantable devices are mistakenly discharged from the hospital.

In order to monitor chronic MI patients and predict the transformation from adaptive remodeling of the heart to maladaptive or adverse remodeling, a reliable molecular imaging probe to assess the extent of fibrosis, particularly at a molecular level, in the myocardium of post-MI patients is needed. In addition, methods for risk stratification of post-MI patients are needed.

### SUMMARY

It is one embodiment of the present invention to provide a radiopharmaceutical compound for imaging of myocardial tissue, the compound having the Formula 1:

E-X—Y Formula 1

wherein E is an A-II receptor binding molecule; X is a bond or a linking moiety; Y is

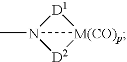

p is an integer from 0 to 3; D¹ and D² are independently

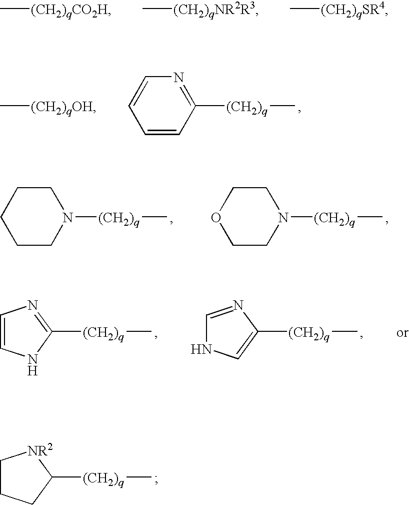

q is an integer from 0 to 3; R² and R³ are independently hydrogen, C₁-C₁₀ alkyl, C₅-C₁₀ aryl, C₁-C₆ hydroxyalkyl, C₁-C₆ acyl, C₁-C₆ aminoalkyl, C₁-C₆ aminoacyl, C₁-C₆ carboxyalkyl, C₁-C₆ carboxyacyl, C₁-C₆ mercaptoalkyl, or C₁-C₆ mercaptoacyl; R⁴ is hydrogen, t-butyl, benzyl, p-methoxybenzyl, C₁-C6 alkoxyalkyl, tetrahydropyranyl, tetrahydrofuranyl, isothiouronium, C₁-C₆ acyl, or C₁-C₆ alkoxycarbonyl; and M is technetium or rhenium.

It is another embodiment of the present invention to provide a method for risk stratification of a post-myocardial infarction (post-MI) patient, the method comprising
(a) imaging myocardium of the post-MI patient using a radiopharmaceutical compound having the Formula 6:

E-X—Y Formula 6

wherein E is an A-II receptor binding molecule; X is a bond or a linking moiety; Y is an imaging moiety; and the compound of Formula 6 is capable of imaging fibrous myocardial tissue;
b) determining an amount of fibrous tissue remote to the MI site of the myocardium to obtain an extent of adverse remodeling, and
c) determining a treatment based on the extent of adverse remodeling.

In still another embodiment, the present invention provides a method for detecting infarcted myocardial tissue comprising imaging myocardium of a post-myocardial infarction patient using a radiopharmaceutical compound having the Formula 1:

E-X—Y Formula 1

Other objects and features will be in part apparent and in part pointed out hereinafter.

### DETAILED DESCRIPTION OF THE EXEMPLARY EMBODIMENTS

Assessment of residual myocardial viability especially for sub-chronic MI and chronic MI patients is a difficult and challenging task. MI results in a myocardium that contains dysfunctional, yet viable regions that overlap with dead cardiomyocytes. The dysfunctional yet viable regions have low blood flow and are shown to be “cold spots” in radioimaging which is indistinguishable from a dead tissue region which is also shown as a “cold spot.” Radiopharmaceuticals currently used in assessing myocardial viability can not differentiate between viable and nonviable zones resulting in the high level of uncertainty in the estimation of myocardial viability. However, radiopharmaceuticals based on specific markers (myofibroblasts) that can target the formation and progression of unwanted fibrous tissues in the myocardium of post MI patients provide important clinical information on the extent of adverse remodeling. It has been discovered that, when such radiopharmaceuticals are used in imaging myocardium of a post-MI patient, the amount of fibrous tissue remote to the MI site of the infarcted myocardium can be determined to obtain an extent of adverse remodeling. A treatment can be determined based on the extent of adverse remodeling.

Thus, in one embodiment, the present invention provides a novel radiopharmaceutical compound for imaging myocardial tissue of an MI patient, the compound having the Formula 1:

E-X—Y Formula 1

wherein E is an A-II receptor binding molecule (targeting moiety); X is a bond or a linking moiety between the targeting moiety and an imaging moiety, and Y is the imaging moiety that emits γ radiation for radioscintigraphic imaging, and is a radionuclide metal carbonyl complex. Specifically, with respect to the radiopharmaceutical composition of Formula 1, X can be any moiety that links the targeting moiety and the imaging moiety without adversely affecting the function of the radiopharmaceutical compound in vivo. For example, X can be —(CH₂)ₘ—, —CONH(CH₂)ₘ—, —NHCO(CH₂)ₘ—, —SO₂NH(CH₂)ₘ—, —SO₂O(CH₂)ₘ—, —SO₂NH(CH₂)ₘ—, —NHCO(CH₂)ₘCONH—, —NH(CH₂)ₘNHCONH(CH₂)ₙ—, —NH(CH₂)ₘNHCSNH(CH₂)ₙ—, —(CH₂)ₘO(CH₂)_{n—, —(CH}₂)ₘS(CH₂)ₙ—, —CH₂)ₘSO(CH₂)ₙ—, and —CH₂)ₘSO₂(CH₂)ₙ—; wherein m and n are integers from 0 to 10. Y is

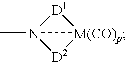

wherein

p is an integer from 0 to 3;

D¹ and D² are independently

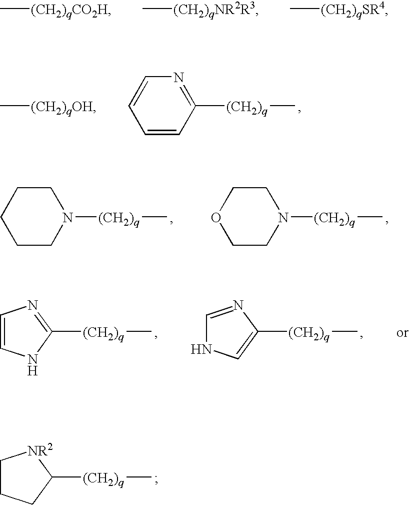

q is an integer from 0 to 3;

R² and R³ are independently hydrogen, C₁-C₁₀ alkyl, C₅-C₁₀ aryl, C₁-C₆ hydroxyalkyl, C₁-C₆ acyl, C₁-C₆ aminoalkyl, C₁-C₆ aminoacyl, C₁-C₆ carboxyalkyl, C₁-C₆ carboxyacyl, C₁-C₆ mercaptoalkyl, or C₁-C₆ mercaptoacyl;

R⁴ is hydrogen, t-butyl, benzyl, p-methoxybenzyl, C₁-C₆ alkoxyalkyl, tetrahydropyranyl, tetrahydrofuranyl, isothiouronium, C₁-C₆ acyl, or C₁-C₆ alkoxycarbonyl; and

M is technetium or rhenium.

In some embodiments, M is technetium-99m. In other embodiments, M is rhenium-186 or rhenium-188.

As mentioned above, E is the targeting moiety that is capable of binding to a myofibroblast. The targeting moiety E can be any compound that exhibits affinity for A-II receptors. In some embodiments, such compound can be a peptide (such as A-II itself or an analog such as saralasin (Sar-Arg-Val-Tyr-Val-His-Pro-beta-Ala)). In other embodiments, it can be a non-peptide compound such as losartan. The compound can have a more pronounced affinity for one type of AII-receptor than for other types (such as losartan) or can exhibit general affinity for all AII receptors. Compounds having more pronounced affinity for particular types of AII receptors (such as AT1 or AT2) may be preferred. Oligopeptides having the motif Arg-Val-Tyr-Ile-His-Pro or an analogous motif in which any of the five amino acid residues are varied either individually or in combination such that the binding to A-II receptor site is preserved are among the preferred peptidic compounds. By way of example, the motifs Arg-Val-Tyr*-XX-His-Pro (where Tyr* is an optionally modified Tyr, eg. Me-Tyr and XX is an amino acid, e.g., Ile or Val) can be used. Other suitable oligopeptides can readily be determined using phage display, combinational chemistry (both peptidic and non-peptidic combinational chemistry), HTS (high throughput synthesis) and CAM.D techniques as is known in the art.

Among non-peptidic compounds, losartan is particularly useful. Other examples of non-peptide compounds include heterocyclic compounds (such as imidazoles, condensed imidazoles, xanthines and pyridones). Examples of non-peptidic compounds are given in WO 91/17148, U.S. Pat. No. 4,355,040, WO 91/18888, WO 91/19715, WO 91/15209, EP-A-420237, EP-A-459136 and U.S. Pat. No. 5,338,744. Furthermore, the development of non-peptidic A-II receptor antagonists is discussed, e.g., by Timmermans et al., in TiPS 12: 55-62 (1991) and Pharm. Rev. 45: 205-251 (1993) and by van Meel et al. in Arzneim-Forsch./Drug Res. 43: 242-246 (1993). Examples include DuP 753 (2-n-Butyl-4-chloro-5-hydroxy-methyl-1-[(2′-(1H)-tetrazol-5-yl)biphenyl-4-yl)methyl]imidazol potassium salt), L-158809 (5,7-dimethyl-2-ethyl-3-[[2′-(1H-tetrazol-5yl)[1,1′]-bi-phenyl-4-yl]-methyl]-3H-imidazo[4,5-b]pyridine), SR-47436 (CAS Registry No. 138402-11-6), OR 117289 (CAS Registry No. 145781-32-4), SKF 108566 (eprosartan), BIBS 39, BIBS 222, ExP 3892 (2-n-propyl-4-trifluoromethyl-1-[(2′-1H-tetrazol-5yl)biphenyl-4-yl)methyl]imidazole-5-carboxylic acid) and CGP 48933 (Valsartan).

Thus, in some embodiments of the present invention, the compounds of Formula 1 are represented by Formulas 2-5:

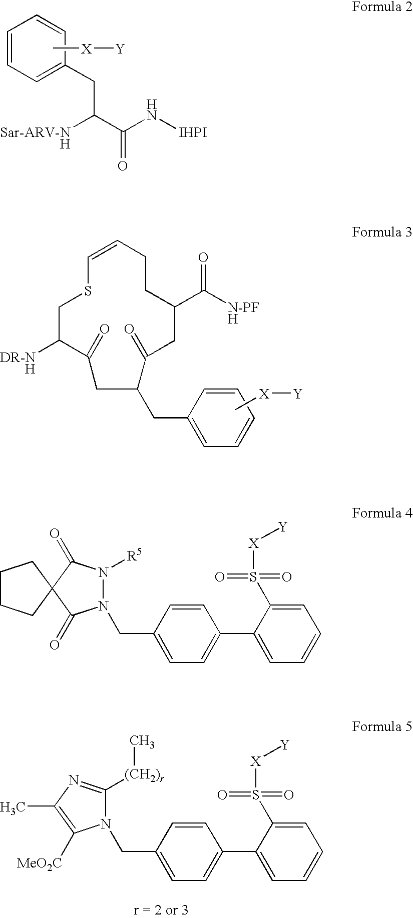

wherein A, D, F, H, I, P, R, and V denote the single-letter designation of a-amino acids, Sar is sarcosine, and R⁵ is methyl or ethyl. X and Y are defined in the same manner as described previously. In some embodiments, X is —CONH(CH₂)ₘ—, —NHCO(CH₂)ₘ—, or —SO₂NH(CH₂)ₘ—. In other embodiments, X is —NH(CH₂)ₘNHCONH(CH₂)ₙ— or —NH(CH₂)ₘNHCSNH(CH₂)ₙ—. In some instances, “m” and “n” are each independently an integer from 0 to 4.

Covalent coupling of the targeting and imaging moieties are well known in the art, and can be effected using linking agents containing reactive moieties capable of reaction with functional groups of the targeting and imaging moieties. The method of coupling these two independent entities to form bioconjugates of the present invention are extensively described in Bioconjugate Chemistry (G. T. Hermanson, Academic Press, New York 1996), which is incorporated herein as reference in its entirety.

Linking can also be effected using enzymes as zero-length crosslinking agents; thus, for example, transglutaminase, peroxidase and xanthine oxidase can be used to produce crosslinked products. Reverse proteolysis may also be used for crosslinking through amide bond formation.

Non-covalent target-imaging moiety coupling can, for example, be effected by electrostatic charge interactions, e.g. through chelation in the form of stable metal complexes or through high affinity binding interaction such as avidin/biotin binding.

So-called zero-length linking agents, which induce direct covalent joining of two reactive chemical groups without introducing additional linking material (e.g. as in amide bond formation induced using carbodiimides or enzymatically) may, if desired, be used in accordance with the invention.

The imaging moiety is a radionuclide metal carbonyl complex, Y, having the following structure:

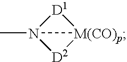

wherein

p is an integer from 0 to 3;

D¹ and D² are independently

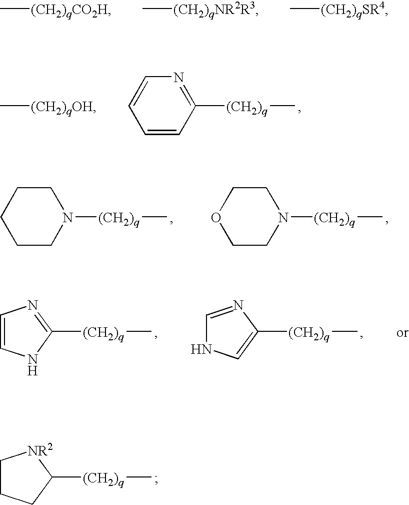

q is an integer from 0 to 3;

R² and R³ are independently hydrogen, C₁-C₁₀ alkyl, C₅-C₁₀ aryl, C₁-C₆ hydroxyalkyl, C₁-C₆ acyl, C₁-C₆ aminoalkyl, C₁-C₆ aminoacyl, C₁-C₆ carboxyalkyl, C₁-C₆ carboxyacyl, C₁-C₆ mercaptoalkyl, or C₁-C₆ mercaptoacyl;

R⁴ is hydrogen, t-butyl, benzyl, p-methoxybenzyl, C₁-C₆ alkoxyalkyl, tetrahydropyranyl, tetrahydrofuranyl, isothiouronium, C₁-C₆ acyl, or C₁-C₆ alkoxycarbonyl; and

M is technetium or rhenium.

In some embodiments, D¹ and D² are independently selected from

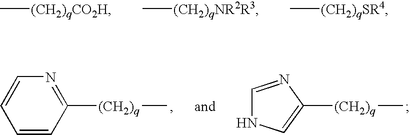

In some embodiments of the present invention, technetium is technetium-99m (Tc-99m). In other embodiments, rhenium is rhenium-186 (Re-186). In still other embodiments, rhenium is rhenium-188 Re-188).

The novel radiopharmaceutical compounds can be used for detecting infarcted myocardial tissue of an MI patient by administering at least one of the compounds to the MI patient and imaging the infarcted myocardial tissue. In some embodiments, the compound is administered intravenously, and is imaged by radioscintigraphy.

The present invention also relates to a method for risk stratification of patients who have suffered at least one myocardial infarction, which is a process for categorization of post-MI patients based on the histopathology of infarcted myocardium. In one embodiment the patient is a human, who has suffered one or more myocardial infarctions. Thus, it is one embodiment of the present invention to provide a method for risk stratification of a post-myocardial infarction (MI) patient, wherein the method comprises

(a) imaging myocardium of the post-MI patient using a radiopharmaceutical compound having the Formula 6:

E-X—Y Formula 6

wherein

E is an A-II receptor binding molecule;

X is a bond or a linking moiety;

Y is an imaging moiety; and the compound of Formula 6 is capable of imaging fibrous myocardial tissue;

b) determining an amount of fibrous tissue remote to the MI site of the myocardium to obtain an extent of adverse remodeling, and

c) determining a treatment based on the extent of adverse remodeling.

Specifically and as discussed above, the radiopharmaceutical compound of the Formula 6 can be a compound of Formula 1 as described above. The risk stratification methods of the present invention can also utilize radiopharmaceutical compounds as described above wherein the imaging moiety Y is a radiohalogen instead of a radionuclide metal carbonyl complex. Any radiohalogen that is capable of imaging infarcted myocardial tissue as known in the art can be used; radioisotopes include ¹²³I and ¹³¹I as well as non zero nuclear spin atoms such as ¹⁸F.

Preferred imaging moieties in some embodiments are metal radionuclides such as ⁹⁰Y, ^{99m}Tc, ¹¹¹In, ⁴⁷Sc, ⁶⁷Ga, ⁵¹Cr, ^{177m}Sn, ⁶⁷Cu, ¹⁶⁷Tm, ⁹⁷Ru, ¹⁸⁸Re, ¹⁷⁷Lu, ¹⁹⁹Au, ²⁰³Pb and ¹⁴¹Ce.

Methods for metallating any chelating agents present are within the level of skill in the art. Metals can be incorporated into a chelant moiety by any one of three general methods: direct incorporation, template synthesis and/or transmetallation. Direct incorporation is preferred by some.

Thus, it is desirable that the metal ion be easily complexed to the chelating agent, for example, by merely exposing or mixing an aqueous solution of the chelating agent-containing moiety with a metal salt in an aqueous solution having a pH in the range of about 4 to about 11. The salt can be any salt, such as a water soluble salt of the metal (e.g., halogen salt), and it may be preferred that such salts are selected so as not to interfere with the binding of the metal ion with the chelating agent. The chelating agent-containing moiety in some embodiments is preferably in aqueous solution at a pH of between about 5 and about 9, more preferably between pH about 6 to about 8. The chelating agent-containing moiety can be mixed with buffer salts such as citrate, acetate, phosphate and borate to produce the optimum pH. Generally, the buffer salts are selected so as not to interfere with the subsequent binding of the metal ion to the chelating agent.

In some embodiments, the use of radioisotopes of iodine, such as 123I and 131I is specifically contemplated.

The imaging moiety can also include organic chromophoric and fluorophoric reporters such as groups having an extensive delocalized electron system, e.g., cyanines, merocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes, etc. Examples of organic or metallated organic chromophores may be found in “Topics in Applied Chemistry: Infrared absorbing dyes” Ed. M. Matsuoka, Plenum, N.Y. 1990, “Topics in Applied Chemistry: The Chemistry and Application of Dyes”, Waring et al., Plenum, N.Y., 1990, “Handbook of Fluorescent Probes and Research Chemicals” Haugland, Molecular Probes Inc, 1996, DE-A-4445065, DE-A-4326466, JP-A-3/228046, Narayanan et al. J. Org. Chem. 60: 2391 2395 (1995), Lipowska et al. Heterocyclic Comm. 1: 427 430 (1995), Fabian et al. Chem. Rev. 92: 1197 (1992), WO 96/23525, Strekowska et al. J. Org. Chem. 57: 4578 4580 (1992), and WO 96/17628. Particular examples of chromophores that may be used include xylene cyanole, fluorescein, dansyl, NBD, indocyanine green, DODCI, DTDCI, DOTCI and DDTCI.

Representative examples of visible dyes for use as the imaging moiety include fluorescein derivatives, rhodamine derivatives, coumarins, azo dyes, metallizable dyes, anthraquinone dyes, benzodifuranone dyes, polycyclic aromatic carbonyl dyes, indigoid dyes, polymethine dyes, azacarbocyanine dyes, hemicyanine dyes, barbituates, diazahemicyanine dyes, stryrl dyes, diaryl carbonium dyes, triaryl carbonium dyes, phthalocyanine dyes, quinophthalone dyes, triphenodioxazine dyes, formazan dyes, phenothiazine dyes such as methylene blue, azure A, azure B, and azure C, oxazine dyes, thiazine dyes, naphtholactam dyes, diazahemicyanine dyes, azopyridone dyes, azobenzene dyes, mordant dyes, acid dyes, basic dyes, metallized and premetallized dyes, xanthene dyes, direct dyes, leuco dyes which can be oxidized to produce dyes with hues bathochromically shifted from those of the precursor leuco dyes, and other dyes such as those listed by Waring, D. R. and Hallas, G., in The Chemistry and Application of Dyes, Topics in Applied Chemistry, Plenum Press, New York, N.Y., 1990. Additonal dyes can be found listed in Haugland, R. P., “Handbook of Fluorescent Probes and Research Chemicals”, Sixth Edition, Molecular Probes, Inc., Eugene Oreg., 1996.

Such chromophores and fluorophores can be covalently linked either directly to the targeting moiety or to or within a linker structure. Once again, linkers of the type described above in connection with the metal imaging moieties may be used for organic chromophores or fluorophores with the chromophores/fluorophores taking the place of some or all of the chelant groups. As with the metal chelants discussed above, chromophores/fluorophores can be carried in or on particulate linker-moieties, e.g. in or on a vesicle or covalently bonded to inert matrix particles that can also function as a light scattering imaging moiety.

The radiopharmaceutical compositions of the present invention can be administered to patients for imaging in amounts sufficient to yield the desired contrast with the particular imaging technique. Where the imaging moiety is fluorescent, generally dosages of from 0.1 micromole to 1.0 millimole of the fluorescent imaging moiety per kilogram of patient body weight are effective to achieve adequate contrast enhancements. Where the imaging moiety is a radionuclide, dosages of 0.01 to 100 mCi, preferably 0. to 50 mCi are normally sufficient per 70 kg body weight.

The compounds of the present invention can be formulated into radiopharmaceutical compositions with conventional pharmaceutical or veterinary aids, for example emulsifiers, fatty acid esters, gelling agents, stabilizers, antioxidants, osmolality adjusting agents, buffers, pH adjusting agents, etc., and may be in a form suitable for parenteral administration. Solutions, suspensions and dispersions in physiologically acceptable carrier media, for example water for injection, are generally preferred. The compounds according to the invention can therefore be formulated for administration using physiologically acceptable carriers or excipients in a manner fully within the skill of the art. For example, the compounds, optionally with the addition of pharmaceutically acceptable excipients, may be suspended or dissolved in an aqueous medium, with the resulting solution or suspension then being sterilized as is known in the art.

For imaging of the heart, the most preferred mode for administering contrast agents is parenteral, e.g., intravenous administration. Parenterally administrable forms, e.g. intravenous solutions, should be sterile and free from physiologically unacceptable agents, and should have low osmolality to minimize irritation or other adverse effects upon administration, and thus the contrast medium should preferably be isotonic or slightly hypertonic. Vehicles include aqueous vehicles customarily used for administering parenteral solutions such as Sodium Chloride Injection, Ringer's Injection, Dextrose Injection, Dextrose and Sodium Chloride Injection, Lactated Ringer's Injection and other solutions such as are described in Remington's Pharmaceutical Sciences, 15th ed., Easton: Mack Publishing Co., pp. 1405-1412 and 1461-1487 (1975) and The National Formulary XIV, 14th ed. Washington: American Pharmaceutical Association (1975). The solutions can contain preservatives, antimicrobial agents, buffers and antioxidants conventionally used for parenteral solutions, excipients and other additives that are compatible with the chelates and that will not interfere with the manufacture, storage or use of products.

In the methods of the present invention, once the radiopharmaceutical compound is administered to a post-MI patient, the imaging is performed using any of the imaging techniques well known in the art. In some embodiments, the imaging is done using single photon emission computed tomography (SPECT), positron emission tomography, optical tomography, optical coherence tomography, or optoacoustic tomography.

The imaging of the patient as described above yields information regarding the amount of fibrous tissue remote to the MI site of the infarcted myocardium. A cardiomyopathic process is initiated by MI, but its progression is related to fibrogenic events, such as myofibroblasts' proliferation and new fibrous tissue formation occurring in noninfarcted myocardium. The appearance of fibrous tissue remote to the MI site and the amount thereof provides important information regarding the extent of adverse remodeling, which in turn allows determination of a suitable treatment. Thus, in addition to imaging the MI site, the methods of the present invention also provide images of the other parts of the myocardium in order to determine the amount and/or progression of fibrous tissue in tissues remote to the MI site. Thus, the methods for risk stratification as described herein provide better insight into choosing the right treatment for a post-MI patient than imaging the primary MI site alone. In some embodiments, the treatment is selected from a ventricular reconstructive procedure, multiple drug therapy, implantation of an implantable device, a percutaneous transluminal coronary artery (PTCA) intervention, or a coronary artery bypass graft (CABG).

In chronic MI patients, the established scar is concordant with high density myofibroblast cells and hence, gives a better signal while unstable scar with a likelihood of infarct expansion appears distorted and continues to expand. Thus, the boundary of the unstable scar can be mapped with a hot and positive signal and can be monitored on a time dependent basis with or without pharmacological intervention. The formation of scar at MI is an important part of tissue repair following heart attack. If a heart attack occurs in the left lower chamber of the heart (left ventricle), for example, over a period of time, the scar tissue can weaken and thin out to become a ventricular ancurism—an abnormal bulge of tissue (unstable scar). This aneurysm, in conjunction with other heart problems, can cause the heart to enlarge thereby reducing its ability to pump blood effectively and simulating a heart failure syndrome. The surgical option to treat such syndromes exhibited by MI patients is interventricular reconstruction surgery where surgeons remove part of the aneurysm scar to reshape the heart, restoring it to normal conical shape. In order for surgeons to carry out the procedure effectively, they need to differentiate an unstable from a stable scar, which can be performed using the imaging methods described herein. While not being bound to a particular theory, it is believed that the unstable scar can be identified through time-dependent infarct expansion of the hot spot image originating from myofibroblasts. Thus, in one embodiment, if a post-MI patient's imaging as described herein reveals an unstable scar and/or a ventricular aneurism, a suitable treatment can be ventricular reconstructive procedure.

In another embodiment, a treatment comprises a multidrug therapy. The drug regime, in general for the treatment of MI patients consists of ACE inhibitors, Angiotensin II receptor antagonist (ARBs), β-blockers, digoxins, aldosterone, diuretics, antifibrotic therapies and combinations thereof. The imaging of a post-MI patient as described herein can provide useful information as to which drugs would be most useful for the patient. Furthermore, the imaging can be performed at regular intervals following the start of the drug therapy (e.g., every six months or once a year) to determine the effects of the drug therapy especially on the appearance of fibrous tissue remote to MI. In one embodiment, such imaging can provide information as to whether the drug therapy is controlling and/or reversing the symptoms. In addition, such imaging can provide information as to whether the dosages should be adjusted, or whether at least one of the drugs should be replaced or discontinued.

In another embodiment, the imaging of a post-MI patient as described herein can identify potential asymptomatic heart failure patients with preserved left ventricular systolic ejection fraction (PLVEF). Left ventricular ejection fraction is an important clinical parameter that is measured in the management of MI patients. However, a weakness of relying on clinical syndromes such as ejection fraction lies in the fact that about ½ to ⅓ of MI patients with heart failure syndrome have normal ejection fraction. These patients are routinely missed in the proper care to avoid a potential heart failure. This often occurs because although the heart pumps properly it fails to fill adequately with blood, a condition of diastolic heart failure. It is well documented that therapeutic interventions aimed solely at correcting a low cardiac output or reduced blood flow do not necessarily slow heart failure (HF) progression. The slow and sporadic spreading of the unwanted fibrous tissue remote to MI is shown to have increased stiffness of the entire myocardium leading to diagnostic heart failure, and the methods of the present invention that image the remote fibrous tissue can be effective in diagnosis of PLVEF. Accordingly, in one embodiment wherein the imaging of a post-MI patient shows PLVEF, a suitable treatment can be an implantable device, such as LVAD.

Left ventricular assist devices (LVAD) are invasive heart assist devices that unload the burden on the heart pumping action and improve the contractile function. However, the high cost of implantation (approximately $196,000) limits the use of LVAD. Currently, LVAD is only used as “a bridge for heart transplantation,” i.e., it is used for heart transplant patients who are waiting on a suitable donor heart to become available. The new molecular biology techniques suggest that myofibroblasts persist or proliferate in repairing tissue as long as the mechanical stress is present. LVADs release mechanical stress, results in the left ventricular unloading (stress relaxation) and has been correlated with regression of myocardial fibrosis. Furthermore, the unwanted accumulation of fibrous tissue remote to MI has been correlated with an increase in the stiffening of the entire myocardium under stress. Thus, the categorization of patients into low risk versus high risk can be based on the estimation of the threshold value of sporadic fibrous tissue appearing in the new areas of myocardium other than the MI site which warrants LVAD implantation.

Percutaneous transluminal coronary angioplasty (PTCA) is also known as coronary angioplasty or angioplasty. It is used to open a blocked artery by inflating a small balloon and inserting a tiny metal structure called a stent to act as permanent scaffolding. The present invention allows the accurate assessment of the residual viability for post MI patients, which in turn categorizes patients into category 1, who benefit from revascularization vs Category 2, who will not benefit from invasive procedure due to high morbidity of revascularization. The success of PTCA can also be valuated from imaging post revascularization patients and comparing the residual viability.

Having described the invention in detail, it will be apparent that modifications and variations are possible without departing the scope of the invention defined in the appended claims. Furthermore, it should be appreciated that all examples in the present disclosure, while illustrating the invention, are provided as non-limiting examples and are, therefore, not to be taken as limiting the various aspects of the invention so illustrated.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above-described aspects and exemplary embodiments without departing from the scope of the invention, it is intended that all matter contained in the above description shall be interpreted as illustrative and not in a limiting sense.

What is claimed is:

1. A radiopharmaceutical compound for imaging of myocardial tissue, the compound having the Formula 1:
E-X—Y Formula 1
wherein
E is an Angiotensin II receptor binding molecule;
X is a bond or a linking moiety;
Y is 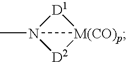
p is an integer from 0 to 3;
D¹ and D² are independently 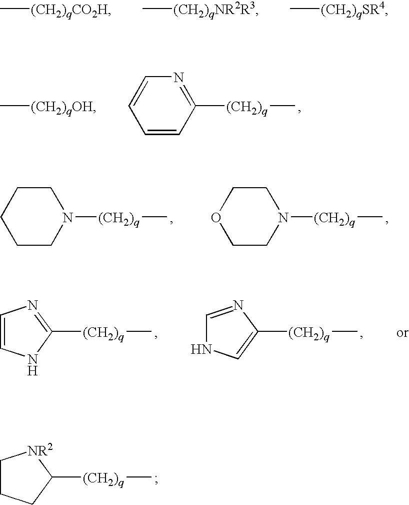
q is an integer from 0 to 3;
R² and R³ are independently hydrogen, C₁-C₁₀ alkyl, C₅-C₁₀ aryl, C₁-C₆ hydroxyalkyl, C₁-C₆ acyl, C₁-C₆ aminoalkyl, C₁-C₆ aminoacyl, C₁-C₆ carboxyalkyl, C₁-C₆ carboxyacyl, C₁-C₆ mercaptoalkyl, or C₁-C₆ mercaptoacyl;
R⁴ is hydrogen, t-butyl, benzyl, p-methoxybenzyl, C₁-C₆ alkoxyalkyl, tetrahydropyranyl, tetrahydrofuranyl, isothiouronium, C₁-C₆ acyl, or C₁-C₆ alkoxycarbonyl; and
M is technetium or rhenium.

2. The compound of claim 1, wherein X is —(CH₂)ₘ—, —CONH(CH₂)ₘ—, —NHCO(CH₂)ₘ—, —SO₂NH(CH₂)ₘ—, —SO₂O(CH₂)ₘ—, —SONH(CH₂)ₘ—,—NHCO(CH₂)ₘCONH—, —NH(CH₂)ₘNHCONH(CH₂)ₙ—, —NH(CH₂)ₘNHCSNH(CH₂)ₙ—, —(CH₂)ₘO(CH₂)ₙ—, —(CH₂)ₘS(CH₂)ₙ—, —(CH₂)ₘSO(CH₂)ₙ—, or —(CH₂)ₘSO₂(CH₂)ₙ—; and m and n are each independently an integer from 0 to 10.

3. The compound of claim 1, wherein the compound is represented by any one of Formulas 2-5, 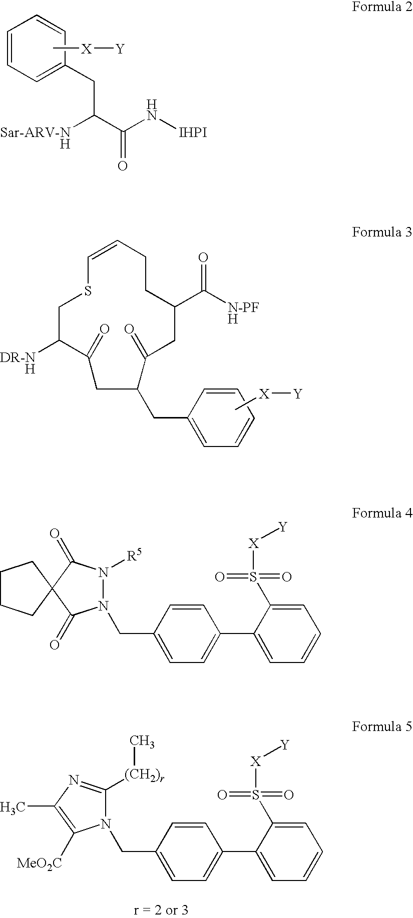
wherein
A, D, F, H, I, P, R, and V denote the single-letter designation of α-amino acids, Sar is sarcosine, and R⁵ is C₁₋₆ alkyl.

4. The compound of claim 1, wherein the compound is represented by Formula 2.

5. The compound of claim 1, wherein the compound is represented by Formula 3.

6. The compound of claim 1, wherein the compound is represented by Formula 4.

7. The compound of claim 1, wherein the compound is represented by Formula 5.

8. A method for risk stratification of a post-myocardial infarction (post-MI) patient, the method comprising:
(a) imaging myocardium of the post-MI patient using a radiopharmaceutical compound having the Formula 6:
E-X—Y Formula 6
wherein
E is an Angiotensin II receptor binding molecule;
X is a bond or a linking moiety;
Y is an imaging moiety; and the compound of Formula 6 is capable of imaging fibrous myocardial tissue;
b) determining an amount of fibrous tissue remote to the MI site of the myocardium to obtain an extent of adverse remodeling, and
c) determining a treatment based on the extent of adverse remodeling.

9. The method of claim 8, wherein the fibrous myocardial tissue is an infarct.

10. The method of claim 8, wherein Y is a fluorescent moiety.

11. The method of claim 8, wherein Y is a radiohalogen.

12. The method of claim 8, wherein Y is 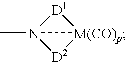
p is an integer from 0 to 3;
D¹ and D² are independently 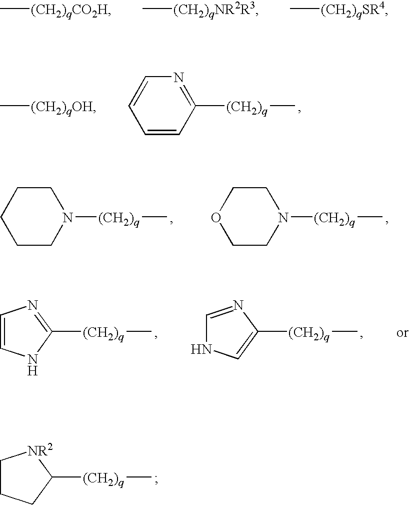
q is an integer from 0 to 3;
R² and ³ are independently hydrogen, C₁-C₁₀ alkyl, C₅-C₁₀ aryl, C₁-C₆ hydroxyalkyl, C₁-C₆ acyl, C₁-C₆ aminoalkyl, C₁-C₆ aminoacyl, C₁-C₆ carboxyalkyl, C₁-C₆ carboxyacyl, C₁-C₆ mercaptoalkyl, or C₁-C₆ mercaptoacyl;
R⁴ is hydrogen, t-butyl, benzyl, p-methoxybenzyl, C₁-C₆ alkoxyalkyl, tetrahydropyranyl, tetrahydrofuranyl, isothiouronium, C₁-C₆ acyl, or C₁-C₆ alkoxycarbonyl; and
M is technetium or rhenium.

13. The method of claim 8 wherein the treatment comprises a ventricular reconstructive procedure, a multiple drug therapy, implantation of an implantable device, a percutaneous transluminal coronary artery intervention, or a coronary artery bypass graft.

14. The method of claim 13 wherein the multiple drug therapy comprises an ACE inhibitors, an Angiotensin II receptor antagonist (ARB), a β-blocker, a digoxin, aldosterone, a diuretic, an antifibrotic therapy or a combination thereof.

15. A method for detecting infarcted myocardial tissue comprising imaging myocardium of a post-myocardial infarction patient using a radiopharmaceutical compound of claim 1.

16. The method of claim 15, wherein M is technetium-99m.

17. The method of claim 15, wherein M is rhenium-186 or rhenium-188.

18. The method of claim 15, wherein the compound is administered intravenously.

19. The method of claim 15, wherein X is —(CH₂)ₘ—, —CONH(CH₂)ₘ—, —NHCO(CH₂)ₘ—, —SO₂NH(CH₂)—, —SO₂O(CH₂)ₘ—, —SONH(CH₂)ₘ—, —NHCO(CH₂)ₘCONH—, —NH(CH₂)ₘNHCONH(CH₂)ₙ—, —NH(CH₂)ₘNHCSNH(CH₂)ₙ—, —(CH₂)ₘO(CH₂)ₙ—, —(CH₂)ₘS(CH₂)ₙ—, —(CH₂)ₘSO(CH₂)ₙ—, or —(CH₂)ₘSO₂(CH₂)ₙ—; and m and n are each independently an integer from 0 to 10.

20. The method of claim 15, wherein the compound is represented by any one of Formulas 2-5, 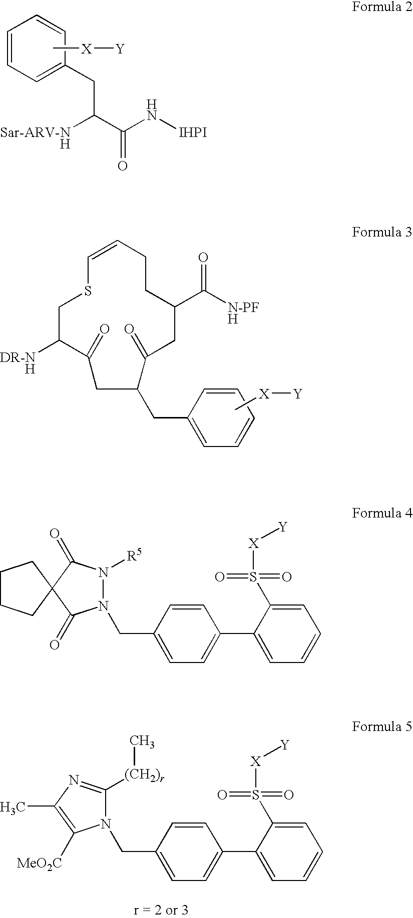
wherein
A, D, F, H, I, P, R, and V denote the single-letter designation of a-amino acids, Sar is sarcosine, and R⁵ is C₁₋₆ alkyl.
